(19) United States
(12) Patent Application Publication
(10) Pub. No.: US 2009/0014002 A1
(43) Pub. Date: Jan. 15, 2009

(54) AIR FILTER ASSEMBLY

(75) Inventors: Brian C. Krafthefer, Stillwater, MN (US); Stephen F. Yates, Arlington Heights, IL (US)

Correspondence Address: HONEYWELL INTERNATIONAL INC., 101 COLUMBIA ROAD, P O BOX 2245, MORRISTOWN, NJ, 07962-2245, US

(73) Assignee: HONEYWELL INTERNATIONAL INC., Morristown, NJ (US)

(21) Appl. No.: 10/907,746

(22) Filed: Apr. 14, 2005

(51) Int. Cl. A61M 16/22

(52) U.S. Cl. 128/205.18

(57) ABSTRACT

A respirator is disclosed that includes a respirator housing, a filtration media element disposed adjacent the respirator housing, and a respiration assist pump adjacent the filtration media element. The respiration assist pump assists the flow of gas through the filtration media element. In addition, a filtration module is disclosed and includes a filtration media element having a photocatalytic agent and a photon source and a gas pump coupled to the filtration media element to assist the flow of gas through the filtration media element. Methods of providing a respirator air stream are also disclosed.

### BACKGROUND

Protection of personnel from toxic or hazardous agents in the air they breathe is an important goal in many fields. Personnel entering a combat zone where chemical and/or biological weapons are a possibility, or responding to attacks in which such weapons have been used currently depends on particulate filtration plus carbon adsorbent technology to avoid inhalation of toxic materials. Other fields in which such protective equipment is used include, for example, those in which personnel handle hazardous chemical materials, including fuel, pesticides, cleaning chemicals, etc.

Carbon cartridges and other similar adsorbent technologies can be an inadequate solution to the problem of personnel protection. They accumulate the toxins within the cartridge during use, and may become saturated if not frequently changed, or they are ineffective against low molecular weight compounds, and they can be ineffective during periods of high humidity. Once close to saturation, they may become a source of these toxins instead of a sink for toxins, and represents a hazardous material which must be disposed of safely. Use of such cartridges also requires the presence of a supply chain to replenish them and dispose of spent cartridges. High Efficiency Particulate Air (HEPA) filters are usually added to carbon cartridges to prevent fouling of the carbon by particulate matter (including biological agents).

### SUMMARY

The present invention relates generally to an improved air filter assembly. In one illustrative embodiment, a respirator is disclosed that includes a respirator housing, a filtration media element disposed adjacent the respirator housing, and a respiration assist pump adjacent to the filtration media element. The respiration assist pump assists the flow of gas through the filtration media element.

In another illustrative embodiment, a filtration module is disclosed. The filtration module includes a filtration media element having a photocatalytic agent and a photon source and a gas pump coupled to the filtration media element to assist the flow of gas through the filtration media element.

Another illustrative filtration module includes a filtration media element having a photocatalytic agent and a plurality of photon sources disposed on or in the filtration media element and illuminating the photocatalytic agent, and a plurality of electrostatic gas pumps coupled to and spaced from the filtration media element to assist the flow of gas through the filtration media element.

Methods of providing a respirator air stream are also disclosed. The illustrative methods includes the steps of providing a respirator housing comprising a filtration media element disposed adjacent the respirator housing, and a respiration assist pump coupled to the filtration media element and adjacent the respirator housing, and pumping respiration air through the filtration media element and into the respirator.

### BRIEF DESCRIPTION OF THE DRAWINGS

FIG. 1 is a schematic front elevation view of an illustrative respirator;

FIG. 2 is a perspective view of an illustrative filtration module shown on the respirator of FIG. 1;

FIG. 3 is a partial cut away perspective view of an illustrative filtration module shown in FIG. 2; and

FIG. 4 is a schematic cross-sectional view of the filtration module shown in FIG. 3.

### DETAILED DESCRIPTION

The following description should be read with reference to the drawings, in which like elements in different drawings are numbered in like fashion. The drawings, which are not necessarily to scale, depict selected illustrative embodiments and are not intended to limit the scope of the invention. Although examples of construction, dimensions, and materials are illustrated for the various elements, those skilled in the art will recognize that many of the examples provided have suitable alternatives that may be utilized.

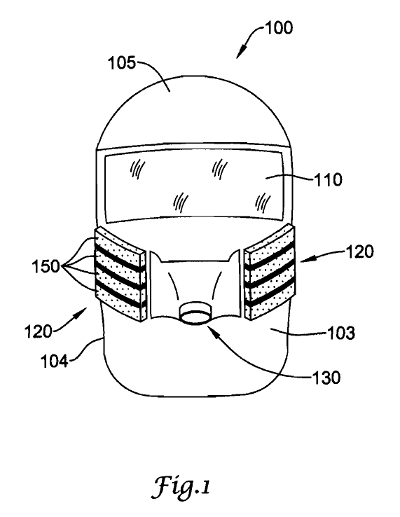

FIG. 1 is a schematic front elevation view of an illustrative respirator 100. The respirator 100 can be useful for removing contaminates from a user's breathing or air supply. One illustrative respirator 100 is shown in FIG. 1, but it is contemplated that the respirator 100 can have any useful form, as desired. In some embodiments, the respirator 100 is a face mask that is capable of being secured to and covers at least a portion of a user's face. In some cases, the respirator 100 may be a helmet covering a majority of a user's head.

The illustrative respirator 100 includes a respirator housing 105. The housing 105 has an outer surface exposed to environmental conditions and an opposing inner surface that is disposed adjacent to a user's face. In some embodiments, the respirator 100 may include a clear face shield 110 disposed within the respirator housing 105. The respirator housing 100 can be formed of any useful material such as, for example, polymeric material.

The illustrative respirator housing 105 may include an air inlet 120 and an air outlet 130. The air inlet 120 can extend through the respirator housing 105 and an air outlet 130 can extend through the respirator housing 105. In some embodiments, the respirator housing 105 includes a two, three, four, or more air inlets 120 and one, two, or more air outlets 130, as desired.

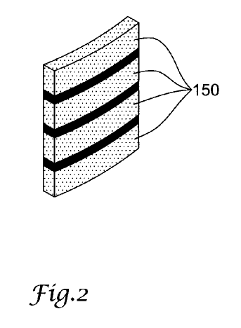

A filtration module, such as filtration module 150, can be coupled to the air inlet 120 or a surface of the filtration module 150 can define the air inlet 120, and can be disposed on or adjacent to the respirator housing 105. In many embodiments, the filtration module 150 forms a unitary article with the respirator housing 105. FIG. 1 illustrates a respirator 100 having two air inlets 120 and a four filtration modules 150 disposed adjacent to each air inlet 120. FIG. 2 is a perspective view of an illustrative filtration module 150 shown on the respirator 100 of FIG. 1.

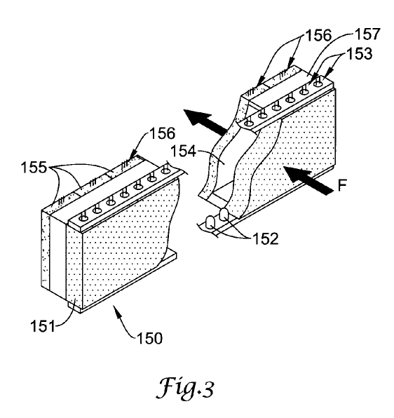

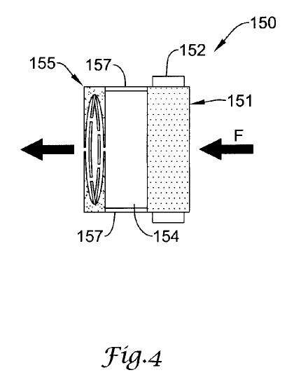

FIG. 3 is a partial cut away perspective view of an illustrative filtration module 150 shown in FIG. 2. FIG. 4 is a schematic cross-sectional view of the filtration element 150 shown in FIG. 3. The illustrative filtration module 150 includes a filtration media element 151 and an adjacent respiration assist pump 155, where the respiration assist pump 155 is in fluid communication with and coupled to the filtration media element 151. In some cases, an air flow cavity 154 is defined between the filtration media element 151, the respiration assist pump 155, and cavity walls 157. In the embodiment shown, the filtration media element 151 is adjacent and coupled or connected to the respiration assist pump 155 via the cavity walls 157. In many embodiments, the filtration media element 151 and respiration assist pump 155 forms a unitary article.

The respiration assist pump 155 can be any useful pump of suitable size. In some embodiments, the respiration assist pump 155 is a diaphragm pump or an electrostatic diaphragm pump. One or more respiration assist pumps 155 can be disposed adjacent the filtration media element 151. In some embodiments, 5 to 500, or 10 to 250, or 25 to 200 or 50 to 150 respiration assist pumps 155 are disposed adjacent the filtration media element 151. The illustrative respiration assist pumps 155 may be disposed adjacent the filtration media element 151 in a two or three dimensional array of respiration assist pumps 155. The illustrative respiration assist pumps 155 are adapted to pump air through the filtration media element 151 and into the interior surface of the respirator housing 105. The respiration assist pumps 155 can pump any useful amount of respiration air through the filtration media element 151 (indicated by the direction of the arrows labeled F). In some embodiments, the respiration assist pumps 155 provide 15 to 30 liters/min of air at a pressure of 1-10 psig. The respiration assist pumps 155 may provide enough air flow and pressure to maintain a positive air pressure within the interior of the respirator 100 while a user of the respirator is breathing. This may help prevent contaminated outside air from leaking into the interior of the respirator housing 105, for example, through seal leaks.

While it is contemplated that the respirator assist pumps 155 can take any suitable form, in some embodiments, the respirator assist pumps 155 can be mesopumps having a single diaphragm as described in U.S. Pat. No. 5,836,750, incorporated by reference herein. Alternatively or in addition, the respiration assist pumps 155 can be a mesopump having a dual diaphragm as described in U.S. Pat. No. 6,179,586, incorporated by reference herein. Also it is contemplated that the respiration assist pumps 155 can include a plurality of such mesopumps in a two dimensional and/or three dimensional array as described in U.S. Patent Publication No. 2004/0020265, incorporated by reference herein. These mesopumps can be manufactured using microelectromechanical systems (MEMS) technology and may operate under electrostatic forces. The mesopumps may include electrical connectors 156 for electrical connection with control electronics (not shown) and/or an electrical energy source such as, for example, a battery (not shown). In some cases, the battery may be disposed on or adjacent to the respirator housing 105, if desired.

Referring to FIG. 3, the illustrative filtration module 150 may include a filtration media element 151. The filtration media element 151 can be formed of any useful filtration media for filtering one or more airborne contaminates. As used herein, the phrase “airborne contaminant” refers to any airborne pollutant or other agent or compound, such as, for example, particulate matter, volatile organic compounds (VOCs), bacteria, pesticides, carbon monoxide, ammonia, hydrogen sulfide, odors, etc. In addition, the phrase “airborne contaminant” can refer to any biological agent such as, for example, viruses, bacteria, spores, and the like. Such biological agents can include human pathogens.

In some cases, the filtration media element 151 may include media that filters particulate matter such as, a HEPA filter. HEPA is an acronym for “High Efficiency Particulate Air.” HEPA filters can capture 99.9% of all particles, including sub-micron sized particles. Alternately or in addition, the filtration media element 151 may include media that filters organic compounds or materials, such as a photocatalytic oxidation filter. The photocatalytic oxidation filter can include a photocatalytic agent disposed on a support structure, and one or more photon sources.

In some cases, the filter module 150 can include a plurality of filtration media elements 151 arranged in series. For example, the filtration module 150 may include a first media element 151 that includes a media that filters particulate matter such as a HEPA filter and a second media element (also labeled 151) arranged to accept air filtered by the HEPA filter. The second media element 151 may include a media that filters organic compound or material, such as a photocatalytic oxidation filter.

Photocatalytic oxidation involves the cleansing of air using a photocatalytic filter. The photocatalytic filter can includes one or more filter media elements coated with a photocatalytic agent. In many embodiments, an ultraviolet lamp can then be used to illuminate the photocatalytic agent, and a catalytic reaction is created when airborne contaminants in the air contact the illuminated photocatalytic agent, causing the airborne contaminant to degrade.

FIG. 3 and FIG. 4 both illustrate the illustrative filtration media elements 151 with integrated or adjacent photon sources 152. Each photon source 152 may be capable of generating a photon or light beam that is directed toward the photocatalytic agent coated on the filter media element 151. The photon sources 152 can be aimed or focused so that the collective light beams substantially or completely cover the photocatalytic agent disposed within or on the filter media element 151.

The photon sources 152 can be a UV light source such as, for example, a light emitting diode and/or a laser emitting diode. The quantity of airborne contaminants that are oxidized per unit of time is proportional to the intensity of the light sources, so increased oxidation can be obtained by using a greater intensity light sources. In some embodiments, the photon sources 152 may be ultraviolet (UV) lamps such as mercury vapor lamps or xenon lamps, UV light emitting diodes (LEDs), or UV laser diodes. In one specific example, the photon sources 152 may be LEDs capable of producing UV light having a wavelength of between about 200 nanometers (nm) and about 400 nm. In various embodiments, the photon sources 152 can be UV LEDs such as model numbers NSHU550A (375 nm), NSHU550B (365 nm), NSHU590A (375 nm), and NSHU590B (365 nm), all manufactured by Nichia Corporation of Japan.

The actual wavelength selected can be dependent upon the adsorption range of the photocatalytic agent. The wavelength of the UV LED can be set so that the UV light is absorbed by the photocatalytic agent. That is, the wavelength of the UV light may be matched to the absorption band of the photocatalytic agent. For example, if the photocatalytic agent is a titanium dioxide having an absorption band of between about 200 nm and 400 nm, then the wavelength of the UV LED can be between about 250 nm and 390 nm. In another embodiment, if the photocatalytic agent is a titanium dioxide having an absorption band of less than about 410 nm, then the wavelength of the UV LED can be less than about 410 nm. Sometimes, a broadband light source may be used, so long as at least part of the spectrum overlaps at least part of the absorption band.

In some embodiments, the photon source 152 includes electrical connectors 153 for electrical connection with control electronics (not shown) and/or an electrical energy source such as, for example, a battery (not shown). In some cases, the battery may be disposed on or adjacent to the respirator housing 105, but this is not required.

In the illustrative embodiment, the photon sources 152 are positioned adjacent to the filtration media element 151 to illuminate the filtration media element 151 with, for example, ultraviolet light and thereby activate the photocatalytic agent on the filtration media element 151, to oxidize airborne contaminates in the air flowing through the filtration media element 151. In some illustrative embodiments, 10 to 100 photon sources 152 may extend along each side of the filtration media element 151 and may extend along a majority of the width of the filtration media element 151, sometimes along the top, bottom, and/or side walls in any configuration to maximize illumination of the filtration media element 151.

As respiration air passes through each filtration module 150, airborne contaminants may become trapped in a particulate filter, when provided, and/or degraded by oxidation with the photocatalytic oxidation filter. Oxidation of an airborne contaminate can occur when an airborne contaminant contacts a portion of the photocatalytic agent that has been activated by the photon source. Increasing a filtration media 151 thickness or surface area containing photocatalytic agent can improve the photocatalytic oxidation filter efficiency. However, this also typically increases the pressure drop across the filtration media element 151 forcing the breathing of the user of the respirator 100 to become less efficient or adds to the stress of the user. Addition of the respiration assist pump 155 counteracts such a pressure drop perceived by a respirator 100 user, allowing the user to breath with less strain. In some cases, the pump 155 can create a positive pressure in the respirator 100 to help prevent contaminates from leaching in through any seals.

In some embodiments, the respirator 100 can include a mechanical particulate filter (HEPA) positioned upstream or downstream of a photocatalytic oxidation filter 150. The mechanical particulate filter functions to remove particulates from an air stream prior to or after the air stream reaches the photocatalytic oxidation filter 150. In other embodiments, a mechanical particulate filter is not included in the respirator 100, or additional mechanical filtering stages can be added, as desired.

Illustrative photocatalytic agents are generally semiconductor materials having a band gap similar in energy to the energies of photons in the visible or UV range. Absorption of light results in the promotion of an electron from the ground state, generating a hole-electron pair. The hole then reacts with adsorbed water to generate hydroxyl radicals.

The size of the band gap required can be determined by the desired wavelength of light. Energy of a photon is inversely proportional to wavelength, and can be specified in units of either Joules/mole, or electron-volts. In many embodiments, the wavelengths corresponding to the following energies:

| Wavelength | Energy (eV) |
|---|---|
| 200 | 6.2 |
| 250 | 5.0 |
| 390 | 3.2 |
| 400 | 3.1 |
| 410 | 3.0 |

In some embodiments, the semiconductor material band gaps are between 2.7 and 4. Examples of useful material include, but are not limited to, titanium dioxide (3.2 eV), tungsten oxide (2.8 eV), strontium titanate (3.2 eV), alpha-Fe2O3 (3.1 eV), zinc oxide (3.2 eV), and zinc sulfide (3.6 eV). In some embodiments, the light sources can emit wavelengths shorter than are required for these band-gaps. Further useful materials include, tantalum oxide, barium titanate (BaTi₄O₉), sodium titanate (Na₂Ti₆O₁₃), zirconium dioxide, cadmium sulfide, K₄Nb₆O₁₇, Rb₄Nb₆O₁₇, K₂Rb₂Nb₆O₁₇, and Pb₁₋ₓK₂ₓNb₂O₆, to list a few. Those materials can be used as a single material or a combination of two or more materials.

Among the above, titanium dioxide is suitable from the viewpoints of the photocatalysis and economical efficiency. There are rutile and anatase types for titanium dioxide. The photocatalytic agent can be a semiconductor metal oxide, more particularly titanium dioxide (in a mixture of the rutile and anatase forms) available under the tradename Aeroxide TiO₂ P25, manufactured by Degussa Chemical Company, Dusseldorf, Germany. In one illustrative embodiment, the photocatalytic agent has a surface area of between about 100-1000 square meters/gram and a thickness of between about 3.0 micrometers and about 5.0 micrometers. The photocatalytic agent can have a relatively large surface area and can be highly active. The photocatalytic agent can be disposed onto a support substrate using conventional methods. Other semiconductive agents that absorb light can also be used, such as, for example, zinc oxide, cadmium sulfide, and zinc sulfide.

Any suitable photocatalytic agent may be disposed on the filtration media element 151. Any suitable material may be used as a substrate material for filter media element 151 such as, for example, a ceramic substrate, an aluminum substrate, an FeCrAlY alloy substrate, and/or a paper/fiber material. Any suitable substrate geometry for the filtration media element 151 may also be used such as, for example, honey-combs, fins, mesh, a filter-type structure, a fibrous type, or a filamentous structure.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

1. A respirator comprising:
a respirator housing having an interior region and an exterior region, wherein when worn by a user, the interior region is exposed to air breathable by the user and the exterior region is exposed to ambient air, the respirator housing further having an air passageway passing from the interior region to the exterior region;
a filtration media element in fluid communication with the air passageway of the respirator housing; and
a respiration assist pump disposed in fluid communication with the filtration media element, wherein the respiration assist pump is an electrostatically actuated diaphragm pump having a pump inlet and a pump outlet with the pump outlet in fluid communication with interior region of the respirator housing, and when electrostatically actuated, assists the flow of gas from the exterior region to the interior region of the respirator housing through the filtration media element, thereby assisting with respiration of the user through the filtration media element.

2. A respirator according to claim 1 wherein the respirator housing has an interior surface and an opposing exterior surface and the respiration assist pump is disposed between the filtration media element and the respirator interior surface.

3-4. (canceled)

5. A respirator according to claim 1 wherein the respirator assist pump comprises a plurality of electrostatically actuated diaphragm pumps.

6. A respirator according to claim 1 wherein the respirator assist pump comprises 25 to 200 electrostatically actuated diaphragm pumps.

7. A respirator according to claim 1 wherein the filtration media element comprises a photocatalytic agent.

8. A respirator according to claim 7 wherein the filtration media comprises an ultraviolet light source.

9. A respirator according to claim 1 wherein the filtration media comprises a particulate filter.

10. A respirator according to claim 6 wherein the filtration media comprises a particulate filter.

11. A filtration module comprising:
a filtration media element comprising a photocatalytic agent and a photon source; and
an electrostatically actuated diaphragm pump coupled to the filtration media element, wherein the electrostatically actuated diaphragm pump, when electrostatically actuated, assists the flow of gas through the filtration media element.

12-13. (canceled)

14. A filtration module according to claim 11 wherein the electrostatically actuated diaphragm pump comprises a plurality of electrostatically actuated diaphragm pumps.

15. A filtration module according to claim 11 wherein the photon source comprises an ultraviolet light source.

16. A filtration module according to claim 11 wherein the filtration media comprises a particulate filter.

17. A method of providing a respirator air stream comprising steps of:
providing a respirator housing having an interior region and an exterior region when worn by a user, wherein when worn by a user the interior region is exposed to air breathable by the user and the exterior region is exposed to ambient air, the respirator housing further having an air passageway passing from the interior region to the exterior region, the respirator housing further having a filtration media element in fluid communication with the air passageway of the respirator housing, and a respiration assist pump fluidly coupled to the filtration media element and adjacent the filtration media element, wherein the respiration assist pump is an electrostatically actuated diaphragm pump having a pump inlet and a pump outlet with the pump outlet in fluid communication with interior region of the respirator housing; and
electrostatically actuating the electrostatically actuated diaphragm pump to pump respiration air from the exterior region to the interior region of the respirator through the filtration media element, thereby assisting with respiration of the user through the filtration media.

18. A method according to claim 17 wherein the pumping step further comprises removing contaminates from the respiration air by pumping the respiration air through the filtration media element.

19. A method according to claim 17 wherein the pumping step comprises pumping respiration air through the filtration media element, to remove organic contaminates from the respiration air, and into the respirator housing.

20. A method according to claim 17 wherein the pumping step comprises pumping respiration air through the filtration media element comprising a photocatalytic agent and a photon source, to remove organic contaminates from the respiration air, and into the respirator housing.

21. A filtration module comprising:
a filtration media element comprising a photocatalytic agent and a plurality of photon sources disposed on or in the filtration media element and illuminating the photocatalytic agent; and
a plurality of electrostatically actuated gas pumps coupled to and spaced from the filtration media element, wherein the gas pump, when electrostatically actuated assists the flow of gas through the filtration media element.

22. A filtration module according to claim 21 further comprising a gas flow channel disposed between the filtration media element and the plurality of electrostatically actuated gas pumps.

23. A filtration module according to claim 21 wherein the plurality of photon sources are disposed on at least two sides of the filtration media element.

24. A respirator comprising:
a respirator housing, wherein the respirator housing has an interior region and an exterior region, wherein when worn by a user the interior region is exposed to air breathable by the user and the exterior region is exposed to ambient air;
a filtration media element disposed adjacent the respirator housing; and
a respiration assist pump disposed between the filtration media element and the respirator housing interior region, wherein the respiration assist pump assists the flow of gas from the exterior region to the interior region of the respirator through the filtration media element, thereby assisting with respiration of the user through the filtration media element;
wherein the filtration media element has an upstream face and an opposing downstream face, with a side extending between the upstream face and the downstream face; and
the filtration media element further having a plurality of spaced Light Emitting Diodes (LEDs) disposed along at least part of the side of the filtration media element that extends between the upstream face and the downstream face, the plurality of LEDs, when activated, shine light into the filtration media element.

25. A respirator comprising:
a respirator housing having an interior region and an exterior region, wherein when worn by a user, the interior region is exposed to air breathable by the user and the exterior region is exposed to ambient air;
a filtration media element; and
an electrostatically actuated respiration assist pump disposed in fluid communication with the filtration media element, wherein the electrostatically actuated respiration assist pump assists the flow of gas from the exterior region to the interior region of the respirator through the filtration media element, thereby assisting with respiration of the user through the filtration media element.

26. A respirator according to claim 1, wherein the respiration assist pump provides a flow of gas from the exterior region to the interior region of the respirator housing to maintain a positive air pressure within the interior region of the respirator.

27. (canceled)

28. A respirator according to claim 1, wherein the electrostatically actuated diaphragm pump includes:
a pump housing having a first electrode; and
at least one diaphragm disposed within the pump housing, the diaphragm having a second electrode, wherein the diaphragm is electrostatically actuated within the pump housing to provide a pumping action by applying a voltage signal between the first and second electrodes.

29. A filtration module according to claim 11, wherein the electrostatically actuated diaphragm pump includes:
a pump housing having a first electrode; and
at least one diaphragm disposed within the pump housing, the diaphragm having a second electrode, wherein the diaphragm is electrostatically actuated within the pump housing to provide a pumping action by applying a voltage signal between the first and second electrodes.

30. A filtration module according to claim 21, wherein the electrostatically actuated gas pump includes:
a pump housing having a first electrode; and
at least one diaphragm disposed within the pump housing, the diaphragm having a second electrode, wherein the diaphragm is electrostatically actuated within the pump housing to provide a pumping action by applying a voltage signal between the first and second electrodes.

31. A respirator according to claim 24, wherein the respiration assist pump is an electrostatically actuated diaphragm pump.

32. A respirator according to claim 25, wherein the respiration assist pump comprises a plurality of electrostatically actuated diaphragm pumps.

33. A respirator according to claim 24 wherein the LEDs are ultraviolet LEDs.

34. A respirator according to claim 24 wherein the side of the filtration media element that extends between the upstream face and the downstream face extends around the perimeter of the filtration media element and includes two opposing segments, wherein at least some of the plurality of spaced Light Emitting Diodes (LEDs) are disposed along each of the two opposing segments.
